(19) United States
(12) Patent Application Publication
(10) Pub. No.: US 2004/0152996 A1
(43) Pub. Date: Aug. 5, 2004

(54) Transformer-isolated alternating current power supply

(76) Inventor: Eberhard Gersing, Goettingen (DE)

Correspondence Address: MARTIN & FERRARO, LLP, 1557 LAKE O'PINES STREET, NE, HARTVILLE, OH, 44632, US

(21) Appl. No.: 10/478,838

(22) PCT Filed: May 23, 2002
(86) PCT No.: PCT/EP02/05665

(30) Foreign Application Priority Data
May 23, 2001 (DE) 101 25 359.1

(51) Int. Cl. A61B 5/05

(52) U.S. Cl. 600/547

(57) ABSTRACT

Medical safety requires for measurement of electrical bioimpedance, wherein an alternating current is applied to the human body, an insulation barrier between the energy source, which provides the energy for the current, and the human being. This insulation barrier can be accomplished by utilizing a transformer. According to the invention, a signal representing the voltage at the output of the current source is used for a positive feedback to ensure that the current provided by the alternating current source is constant over a wide range of loads Z_{X} and a wide range of frequencies. For instance, the alternating current applied to the human body by a transformer is measured with the help of a current measuring transformer and the measured signal utilized for controlling in a closed loop the alternating voltage at the input of the current source, which drives the transformer for the purpose of generating the alternating current.

The invention is related to an alternating current source of floating type, which is applicable for the measurement of electrical impedance or admittance, in particular at the human body.

The measurement of complex electrical impedance or admittance at biological tissues, for instance, the human body, reveals information about their state in the event of application of appropriate frequencies. For example, impedance measurements performed at the heart before, during and after cardiac surgery provide valuable information for diagnosis of the patient suffering from heart failure. Impedance monitoring during organ transplantation determining the damage to the organ because of ischemia and/or its resuscitability may serve as another example.

Invasive electrical measurements applied to internal human organs are subject to restrictive requirements which ensure medical safety (IEC 60601-1). In particular, the leakage current flowing across the human body is limited to 10 μA (micro Amps). Furthermore, the insulation between the AC powered measurement instruments and the human body must withstand voltages of up to 4 kVᵣₘₛ (4,000 Volts root-mean-square). Common impedance analyzers, intended, but not limited, for use in communication electronics, don't meet these requirements for medical safety.

Impedance measurement is performed using a setup of two or four poles. Two-polar setup requires placement of two electrodes at the human body. These two electrodes serve for injection of the alternating current as well as for the sensing of the resulting voltage. Two-polar application allows one-step measurement of current and voltage by the same measurement unit, resulting in a straight forward determination of impedance (or admittance).

Four-polar setup distinguishes between two electrodes, which are used for current injection, and two separate electrodes for voltage sensing. Alternating current source and voltmeter may be separated. In order to determine the impedance by means of the sensed voltage, the magnitude and phase of the alternating current applied must be known, i.e., an alternating current of known magnitude and phase must be generated, or it must be measured. Commonly the applications for impedance measurements require measurements in a wide range of frequencies, for instance, 1 KHz to 1 MHz, at various load impedances.

Thus, requirements for the alternating current source intended for use in biomedical applications are tough. The current source must not only meet the aforementioned requirements for medical safety but also generate a current of constant amplitude at a wide spectrum of frequencies.

State of the art is to utilize transformers, which defines a first circuit applied to the patient and second circuit, which is insulated from the first circuit, and connected to the measurement device, which may include AC powered electrical components, such as the current generator.

A design utilizing simple transformer coupling limits the usable frequency spectrum at the output of the current source and the impedance of the current source itself (caused by the non-ideal properties of a real transformer). As a result, the requirement of providing a current of constant amplitude at a wide spectrum of frequencies and at a variety of load impedances can not be met.

The invention describes an alternating current source of floating type, which generates a current of constant amplitude at various loads, and which is used preferably for a wide frequency spectrum, such as 1 KHz to 1 MHz.

This task is accomplished, according to the invention, by a feedback of a signal to the generator circuit in the measurement device, which incorporates an alternating current source, which generates a current which is injected into the human body using a transformer, which defines a patient side, and a device side, which is insulated from the patient side, with the transformer coil on the patient side leading to terminals which are connected to the human body, and with the alternating current source including a generator on the device side, which generates an alternating voltage signal or alternating current signal, which in turn produces an alternating voltage or alternating current at the patient side in the event both terminals are connected through the human body as the load, with the feedback of a signal to the device side established in such a way that the alternating voltage applied to the device side of the transformer is increased according to an increase in load voltage. As a result, the inner impedance of the current source is increased artificially, providing a constant current output at various loads and current frequencies.

According to a first embodiment of the invention, an alternating current source of the aforementioned type is modified in such a way that a voltage measured at a portion of the coil of the device side is used as a positive feedback. This embodiment is simple and, thus, of low cost.

According to another embodiment of the invention, a first end of the transformer coil located on the device side is connected to the inverting input of a differential amplifier, and a second end of the coil in connected to the output of the differential amplifier and, via a voltage divider, to the non-inverting input of the differential amplifier.

According to another embodiment of the invention, a first end of the a first transformer coil located on the device side is connected to the inverting input of a differential amplifier, and a second end of the first coil in connected to the output of the differential amplifier, and, in addition to the first coil, at least a second coil (encompassing the same transformer core) located on the device side, with the second coil not connected to the generator but with the non-inverting input of the differential amplifier. Accordingly, a voltage induced in the second coil is provided to the input of the differential amplifier. According to this embodiment, a voltage is induced in the second coil, which, when used for feedback, accounts indirectly for the non-ideal properties of the transformer (core losses, stray inductivities).

According to another embodiment of the invention, the alternating current source encompasses a means for transforming the alternating current at the patient side, while maintaining the insulation barrier, into a control signal at the device side, which controls the alternating voltage signal provided by the generator. This control signal represents the alternating current of the patient side and, ideally, a direct measurement of the current. This embodiment offers the advantage of a closed loop control of the current source because of the current measurement and the feedback of related signal. Thus, the stability of the current source output is improved significantly.

The means for transforming may be a second transformer. This solution is suitable because the alternating current source incorporates a first transformer already. After the transformer the current induced on the device side is converted into a voltage utilizing a current-voltage-converter (for instance, an operational amplifier with a resistor in the feedback path), with the voltage being a direct measure for the current output of the alternating current source.

Instead of utilizing a second transformer, a voltage related to the alternating current on the patient side can be obtained by utilizing a magnetic field sensor, which makes use of the magnetic field caused by the current. The magnetic field, for example, changes the resistance of a resistor, which is sensitive to magnetic fields and which is embedded with a direct current (DC) circuitry; the voltage across the resistor serves as a control signal for the alternating current source. An alternative is the utilization of a Hall sensor, upon which, when subject to a DC current, the so-called Hall voltage is established, which is depending on the magnetic field, and which serves as a control signal.

The aforementioned embodiments obtain a voltage, which relates to the output of the current source. Alternatively, the control may be established by obtaining a current signal.

The voltage obtained can be used for controlling in the following way: the voltage is compared directly with the alternating voltage provided by the generator, for instance, by using a differential amplifier. The voltages are compared either directly or by implementing a resistor bridge. The differential signal is fed to one input (for example, the inverting input) of an operational amplifier, while the other input (for example, the non-inverting input) is connected to ground. The transformer circuitry serves as a voltage source in the event that one end of the transformer coil on the device side is connected to the output of the amplifier, while the other end is connected to ground. If the other end is connected to the summation point (inverting input of the operational amplifier) instead to ground, then the circuitry serves as a current source.

This embodiment has the advantage that only a few active components other than the generator are required. Thus, it represents a good compromise between simplicity of the efforts and the advantage of true close loop control of the current output of the current source.

More efforts can be made to make the system less sensitive to the frequency-dependent phased shifts caused by its components and load conditions, thus making the new alternating current source applicable over wider frequency ranges. Therefore, the control signal (actual current) is fed through a rectifier followed by a low pass filter. In addition, a direct current (DC) source is needed, which provides a constant voltage. Then the rectified control signal (actual) is compared with this constant voltage (reference) by providing these signals as inputs to a differential amplifier. The amplified differential signal serves for controlling the alternating voltage signal provided by the generator. For example, the amplified differential signal is put towards a multiplier, which multiplies the alternating voltage provided by the generator with the control signal. Alternatively, the multiplier can be replaced by a voltage controlled amplifier, which utilizes the amplified differential signal as a control signal. Alternatively, resistors, which are controllable electrically, magnetically or optically, may be used.

With the later embodiment being the most costly, it generates a constant current applicable over an extended frequency range and for an extended range of loads.

Preferred embodiments of the alternating current source according to the invention are described in the following figures.

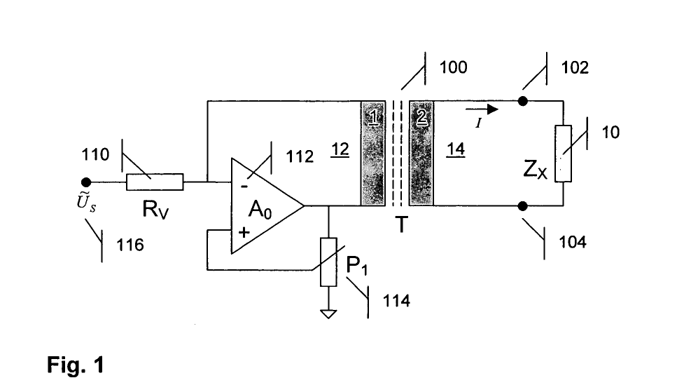

FIG. 1 First embodiment of the alternating current source;

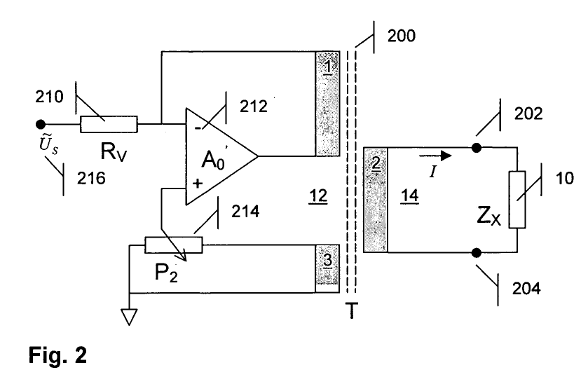

FIG. 2 Second embodiment of the alternating current source;

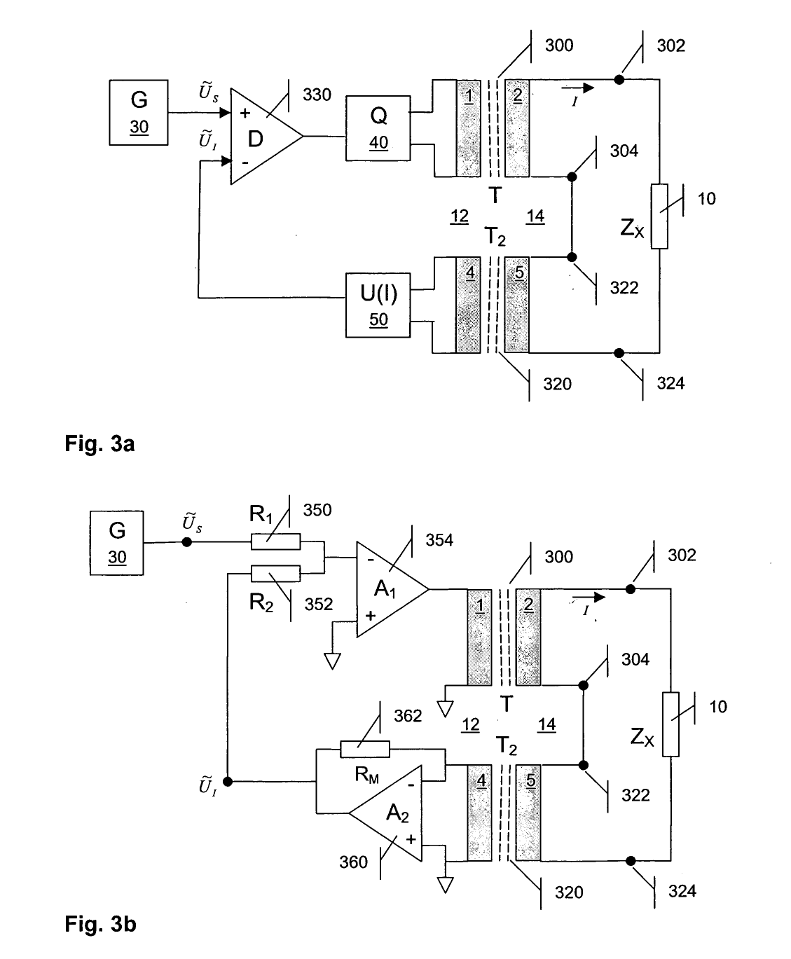

FIG. 3a Third embodiment of the alternating current source in a general form,

FIG. 3b Third embodiment of the alternating current source in a specific form;

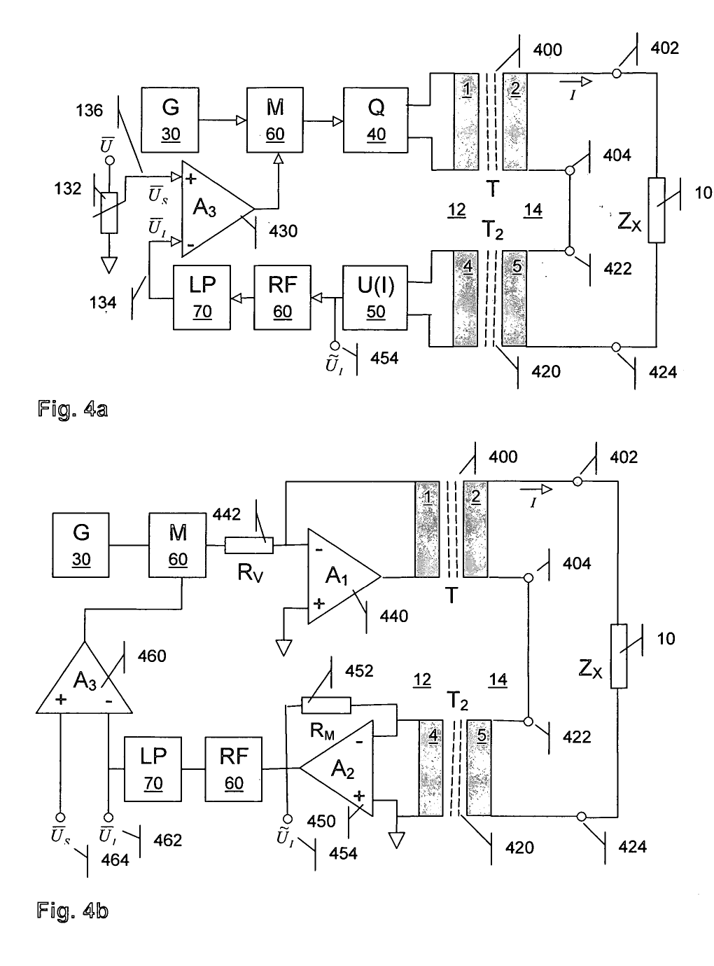

FIG. 4a Forth embodiment of the alternating current source in a general form;

FIG. 4b Forth embodiment of the alternating current source in a specific form;

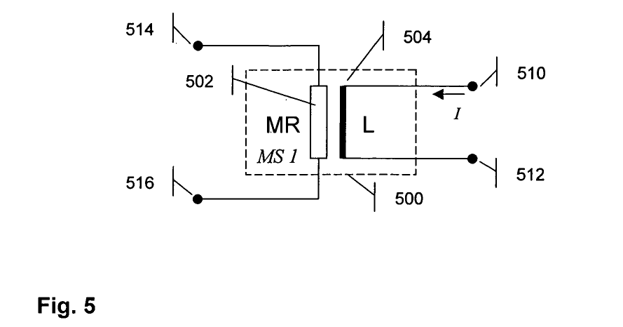

FIG. 5 Schematic design of a magnetic field sensor circuit featuring a magnet field sensitive resistor, which is used to convert a current into a voltage while keeping the potentials isolated;

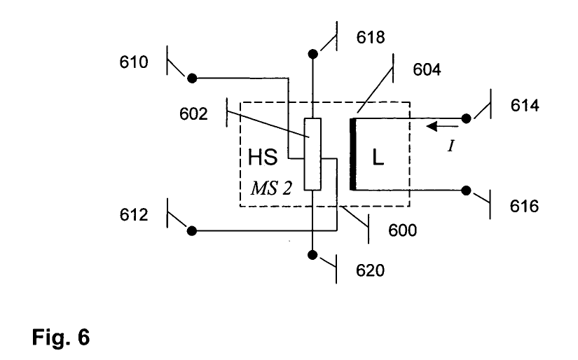

FIG. 6 Schematic design of a magnetic field sensor circuit featuring a Hall sensor, which is used to convert a current into a voltage while keeping the potentials isolated,

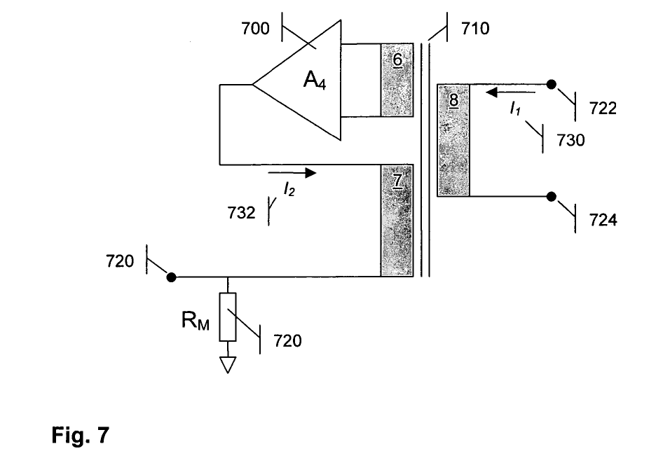

FIG. 7 Schematic design of a preferred embodiment of the current-to-voltage converter U(I) described in FIG. 3a and FIG. 4a.

FIG. 1 illustrates an alternating current source, where a transformer T 100 establishes the potential isolation required for medical safety between an AC powered device side 12 and a patient side 14 (right), whereof a transformer coil 2 at the patient side 14 provides the two terminals 102 and 104, which are connected via the load Z_{X} 10 representing the patient. Furthermore, the alternating current source encompasses a generator (not shown), which generates an alternating voltage signal Ũₛ 116. This alternating voltage signal Ũₛ 116 is converted into an alternating current via a resistor R_{V} 110 and an amplifier A₀ 112 (preferably an operational amplifier), whereof the alternating current excites a first transformer coil 1 on the device side 12 in such a way that an alternating current is generated on the patient side 14.

The terminal of the resistor R_{V} 110, which is connected to the first terminal of the first transformer coil 1 on the device side 12, is also connected to the inverting input of the differential amplifier A₀ 112. The second terminal of the first transformer coil 1 on the device side 12 is connected to the output of the differential amplifier A₀ 112 and, via a potentiometer P₁ 114, to the non-inverting input of the differential amplifier A₀ 112.

Thus, the voltage established at the output of amplifier A₀ 112 on the device side 12 is utilized for a positive feedback to the alternating current source. This causes the impedance of the current source to increase, resulting in an extended range of loads Z_{X} 10, where a current of constant amplitude is maintained. The potentiometer P₁ 114 is used to set the degree of feedback. If required, the positive feedback is designed to be frequency dependent in order to establish for the alternating current source a wider range of applications.

A further improvement is accomplished with the embodiment illustrated in FIG. 2 utilizing a transformer T 200. In addition to the first transformer coil 1 on the device side 12, a second transformer coil 3 on the device side 12 is set up, which is not connected to the first transformer coil 1 but, for example, wrapped around the same transformer core. Electromagnetic induction induces a voltage in the second transformer coil 3 on the device side 12, whereas the second coil 3 may consist of one or more windings. Different to the alternating current source of FIG. 1, where the voltage across the first coil 1 on the device side 12 is feed back, the voltage across the second coil 3 on the device side 12 is fed (with the correct phase) to the non-inverting input of the differential amplifier A_{O} 212. Again, a potentiometer P₂ 214 may be used to set the degree of feedback.

The terminals 202, 204 of the transformer coil 2 at the patient side connect to the load Zₓ 10 representing the patient.

Compared to the embodiment of FIG. 1, the embodiment of the alternating current source of FIG. 2 has the advantage that, instead of directly feeding back the voltage at the output of amplifier A₀ 212, a detour is made via the coil 3 of the transformer T 200. In this embodiment, the feedback accounts for the properties of the transformer T 200, too.

The aforementioned embodiments (FIGS. 1, 2) are based on the positive feedback of voltages. More advantageous is the implementation of a true current control in a closed loop, which measures the output current, feeds back a signal representing the measured current (while maintaining the potential isolation), and uses this feedback signal for control.

The embodiment according to FIG. 3a and FIG. 3b introduces a first transformer T 300 and a second transformer T₂ 320. A generator 30 establishes an alternating voltage signal Ũₛ (of a desired frequency and amplitude) at the non-inverting input of the differential amplifier D 330.

The output the differential amplifier D 330 is connected with the input of a voltage controlled current source Q 40, which feeds the primary coil 1 (at the device side 12) of the first transformer T 300. At the patient side 14 one end of the coil 2 of the first transformer T 300 is connected via the terminal 302 with the load Z_{X} 10 representing the patient. The other end of the coil 2 of the first transformer T 300 is connected with the terminal 304.

The other end of the load Z_{X} 10 is connected via the terminal 324 with the coil 5 (on the patient side 14) of the second transformer T₂ 320, i.e., the current measuring transformer. The other end of coil 5 of transformer T₂ 320 is connected via the terminal 322 to terminal 304 closing the circuit on the patient side 14.

The coil 4 on the device side 12 of the second transformer T₂ 320 is connected to a current-to-voltage converter U(I) 50. Its output, a voltage proportional to the current I through the load Z_{X} 10 (patient), is fed (as the actual value Ũ_{I}) to the inverting input of the differential amplifier D 330, closing the control loop.

FIG. 3a shows the second transformer T₂ 320 of the same size as the first transformer T 300, but T₂ 320 may in fact be significantly smaller than T 300 because of the smaller amount of power to be transformed. The coil 4 on the device side 12 of the transformer T₂ 320 is practically short-circuited by the function of the current-to-voltage converter circuit U(I) 50 (consisting of an operational amplifier A₂ 360 and a resistor 362, like in FIG. 3b), thus defining the function of the second transformer T₂ 320 being that of a current measuring transformer.

FIG. 3b shows a circuit exhibiting a similar function like in FIG. 3a. Whereas in FIG. 3a the difference between the “set point” voltage Ũₛ from the generator G 30 and the actual value of the current proportional voltage Ũ_{I} is amplified in the differential amplifier D 330 and converted into a proportional current by the subsequent current source Q 40, in FIG. 3b the mentioned difference is provided by the half-bridge consisting of the resistors 350 and 352 and amplified by the amplifier A₁ 354 driving finally the coil 1 (on the device side 12) of the transformer T 300.

The current delivered by its coil 2 on the patient side 14 to the load Z_{X} 10 (patient) and flowing through the coil 5 on the patient side 14 of current transformer T₂ 320 is converted into a voltage Ũ_{I} (actual value, proportional to the current in the patient circuit) by the circuit consisting of the coil 4 of the transformer T₂ 320, the operational amplifier A₂ 360 and the feedback resistor 362.

FIG. 4a and FIG. 4b describe a further improvement of the embodiment of the alternating current source of FIG. 3a and FIG. 3b. Like in FIG. 3a and FIG. 3b, on the patient side 14 a coil 2 of a first transformer T 400 is connected in series with a coil 5 of the second transformer T₂ 420 by connecting the terminal 404 of the first transformer 400 with the terminal 422 of the second transformer 420. The load Z_{X} 10 which represents the patient is connected with the terminal 402 of the first transformer 400 and with the terminal 424 of the second transformer 420.

According to the new embodiment (FIGS. 4a, 4b), the signal Ũ_{I}, at the output of the current-to-voltage converter circuitry U(I) 50, is a measure of the output current of the alternating current source. It is fed through a rectifier RF 60 followed by a low pass filter LP 70. The rectified voltage signal \overline{U}_{I} 134 is compared with the preset voltage signal \overline{U}ₛ 136, i.e., each signal is an input to a differential amplifier A₃ 430. The preset voltage signal \overline{U}ₛ 136 is generated by a DC voltage source \overline{U} (not shown) and set via a potentiometer 132.

The output of the differential amplifier A₃ 430 is fed to a controllable circuit, for example, but not limited to, a multiplier M 60. The multiplier M 60 takes into account the alternating voltage signal established by the generator 30. The multiplier M 60 multiplies the two input signals. Thus, the alternating voltage at the output of the multiplier M 60 is amplified or attenuated depending on the difference between \overline{U}ₛ 136 and \overline{U}_{I} 134.

The alternating voltage signal at the output of the multiplier M 60 controls a current source Q 40. The current source 40 can be designed in such a way that it establishes, in conjunction with the coil 1 of the first transformer T 400, a self consistent voltage controlled current source. Alternatively, Q can be designed as a voltage source. For example, amplifier A₁ 440 (FIG. 4b) with input resistor 442 and a feedback resistor instead of the transformer coil 1 in the feedback path, drives the coil 1 (on the device side 12) of transformer T 400. In this case, the other lower end of the coil 1 of transformer T 400 must be grounded. Finally, the total circuit acts as a current source because of the feedback loop established by the current measuring circuit consisting of transformer T₂, current-to-voltage converter U(I) 50, rectifier RF 60 and low-pass filter LP 70.

The embodiments of FIGS. 4a and 4b stabilize the output current I despite a large variation of the load Z_{X} 10—by closed loop control. This embodiments are used preferably for a wide range of frequencies of the current output I because the control is not accomplished by signals alternating at the measurement frequency but DC signals. Accordingly, sufficient stability is gained also at high frequencies.

According to the embodiments illustrated in FIG. 3a, FIG. 3b, FIG. 4a and FIG. 4b, the output of the alternating current source is measured by a second transformer T₂. There are other ways to obtain a voltage signal which is representative to the alternating current on the patient side. The requirement of maintaining an insulation barrier withholding 4 KV_{RMS}, however, remains. A possible solution is the use of magnetic field sensors. According to the principle of magnetic field sensors, the alternating current to be measured generates a magnetic field with a magnitude proportional to the current magnitude, which causes a change to the sensor and, in turn, a voltage signal.

Types of magnetic field sensors are resistors sensitive to magnetic fields. A special kind MR of these resistors, which utilize the “giant magnetic resistance effect”, also referred to as GMR, shows very high sensitivity.

An embodiment incorporating such kind of resistor illustrates FIG. 5. The current of measure enters the magnetic field sensor (dashed lines) between the terminals 514, 516 and flows through a conductor L 504. A resistor MR 502 sensitive to magnetic fields is located closely to but insulated from this conductor L 504. A constant DC current applied between the terminals 510, 512 flows through this resistor MR 502. Simultaneously the voltage across the terminals 510, 512 is measured. The alternating current I through the conductor L 504 causes a corresponding change in the related magnetic field, and, in turn, in the voltage drop between the terminals 510, 512. The resulting voltage signal can be used as a control signal. With regards to the aforementioned embodiments, this circuitry MS 1 502 replaces the transformer T2 plus the subsequent current-to-voltage converter. The (alternating) voltage obtained between the terminals 514, 616 corresponds to the voltage Ũ_{I} of FIG. 4b, FIG. 3a und FIG. 3b.

In order to operate the embodiment of FIG. 5 in the linear range, a bias magnetic field must be provided, i.e. a certain level of magnetic field at the resistor MR 502, which is established, for example, by a permanent magnet (not shown) or by a separate conductor or coil carrying a suitable DC current (not shown).

FIG. 6 describes a magnetic field sensor MS 2 600 incorporating a conductor L 604 subject to the alternating current of measure, which is applied to terminals 614, 616. A Hall Sensor HS 602 is located closely to but insulated from this conductor L 504. This Hall sensor HS 602 utilizes the well-known effect according to which a magnetic field perpendicular to the current flow in a conductor causes a transverse voltage (Hall Voltage) to occur, which is proportional to the magnetic field applied. Accordingly, an auxiliary DC current is applied between terminals 618, 620 of the Hall Sensor HS 602 in order to get the magnetic field dependent Hall Voltage between the terminals 610, 612.

Regarding the embodiment of FIG. 4b, FIG. 3a and FIG. 3b, this Hall voltage takes the place of Ũ_{I}.

Instead of using a second transformer T₂ or magnetic field sensors, a voltage signal representing the alternating current at the patient side can be obtained via an optocoupler, which is designed to withstand 4KV_{RMS}. An optocoupler consists, for example, of a photodiode, which is located on the patient side, and a phototransistor, which is part of the device circuitry. Because commonly available linear optocouplers are designed only for one current direction, the emitting diode must be supplied with a (constant) offset current, which keeps the optocoupler in the center of the linear operating range. Utilizing the aforementioned setup, a signal can be obtained at the device side which is proportional to the alternating current on the patient side. The provision of the control current and the offset current for the optocoupler on the patient side requires active electronic components at the patient side, a disadvantage considering the safety barrier and the maximum allowed leakage currents the patient is be subject to.

According to the specific embodiments of FIG. 3b and FIG. 4b, the current-to-voltage converter consists of an operational amplifier A₂ and a resistor R_{M} in the feedback path. Other implementations of a current-to-voltage converter are possible.

FIG. 7 illustrates a specific implementation of the current-to-voltage converter. In this embodiment the second transformer 710 features two coils at the device side, a first coil 6 and a second coil 7. The first coil 6 is connected to the input of a differential amplifier A₄ 700. The output of the amplifier A₄ 700 is connected to one end of coil 7. The other terminal of the coil is connected via a resistor R_{M} 720 to ground. According to the embodiment of FIG. 7, the current-to-voltage conversion performs as follows: An alternating current I₁ 730, which flows through a coil 8 on the patient side of the current measuring transformer 710, excites a corresponding magnetic flux in the core of the transformer 710 which induces a corresponding voltage in the first coil 6 at the device side, which drives the amplifier A₄ 700 (with high open loop gain) and causes at its output a current I₂ 732, which flows through the second coil 7 of the transformer 710 and compensates with the magnetic flux generated by itself the magnetic flux generated by the current I₁ 730. The current I₂ 732, which is then proportional to the current I₁ at the patient side, causes a voltage drop across RM proportional to the current I₁ 730, which is used as a control signal, similar to the control signal in the embodiments according to FIG. 3a, FIG. 3b, FIG. 4a and FIG. 4b.

A number of the aforementioned circuitries within the embodiments can be replaced with similar operating circuitries. Instead of a multiplier M a different controllable component can be used, for instance, a voltage controlled amplifier or a linear optocoupler, also a electrically controllable resistor (for example, field effect transistor) or a magnetically or optically controllable resistor.

The invention provides a floating alternating current source, which meets the requirements for medical safety and, at the same time, generates an alternating current, of which the amplitude is stable over a wide frequency range, for instance, 1 KHz to 1 MHz, at various loads.

1. An alternating current source for generating an alternating current (I) across the body of a patient (Z_{X}), comprising
A first transformer, which defines a patient side and a device side which is insulated from the patient side, and whose coil at the patient side leads to terminals, which can be connected to the patient, and
A generator on the device side, which generates an alternating voltage or an alternating current, and which excites a coil at the device side in such a way that an alternating current is generated at the patient side upon connecting the terminals via the patient as the load (Z_{X}),
Means for converting the alternating current at the patient side into a control signal, which is accessible at the device side and isolated from the patient side, and which controls the signal generated by the generator.

2. The alternating current source of claim 1 (FIGS. 3a, b & FIGS. 4a, b),
wherein said means is a second transformer (T₂).

3. The alternating current source of claim 2 (FIGS. 3a, b & FIGS. 4a, b),
wherein at the device side the second transformer is followed by current-to-voltage converter, and wherein the control signal provided at the output of the current-to-voltage converter is an accessible voltage (Ũ_{I}).

4. The alternating current source of claim 3 (FIGS. 3b & FIG. 4b),
wherein the current-to-voltage converter incorporates an operational amplifier (A₂) and a resistor (R_{M}) in the feedback path.

5. The alternating current source of claim 2 (FIG. 7),
wherein the second transformer incorporates at the device side a first coil and a second coil, wherein the first coil is connected to the input of an amplifier (A₄), whose output is connected to the second coil in such a way that an amplified current flows as a compensatory current through the second coil and a resistor connected to the second coil, with a control signal (Ũ_{I}) obtained at the resistor.

6. The alternating current source of claim 1 (FIG. 5),
wherein said means incorporates a conductor (L) subject to the alternating current at the patient side, and a resistor (MR) sensitive to magnetic fields, which is located in proximity of but insulated from the conductor (L),
wherein a constant direct current, which is generated by a direct current source incorporated into the device, is applied to the resistor (MR),
wherein a magnetic bias is provided (by an additional conductor or coil carrying a suitable DC current or a permanent magnet) in order to enable linear signals of both polarities,
and wherein the voltage drop across the resistor (MR) depending on the magnetic field defines a control signal (actual value of the current).

7. The alternating current source of claim 1 (FIG. 6),
wherein said means incorporates a conductor (L) subject to the alternating current at the patient side, and a Hall Sensor (HS), which is located in proximity of but insulated from the conductor (L),
wherein a direct current, which is generated by a direct current source incorporated into the alternating current source, is applied to the Hall Sensor (HS),
and wherein the transversal voltage across the Hall Sensor (MR) defines a control signal (actual value of the current).

8. The alternating current source of one of the claims 3, 5, 6 or 7,
wherein the control signal (Ũ_{I}) is compared to a preset alternating voltage (Ũₛ) which is provided by the generator,
and wherein the difference between the control signal (Ũ_{I}) (actual value of the current) and the preset alternating voltage (Ũₛ) is amplified and the amplified differential signal excites the first transformer coil at the device side.

9. The alternating current source of claim 8,
wherein two resistors connected in series (R₁, R₂) are provided
and wherein the alternating voltage (Ũₛ) generated by the generator is applied to the first resistor (R₁) and the control signal (Ũ_{I}) (actual value of the current) is applied to the second resistor (R₂),
and wherein the voltage at the connection of both resistors is an input to an amplifier (A₁), whose output provides the alternating current for the first transformer at the device side.

10. The alternating current source of one of the claims 3, 4, 5, 6 or 7,
wherein a rectifier (RF) rectifies the control signal (Ũ_{I}) (actual value of the current).

11. The alternating current source of claim 10,
wherein a low pass filter (LP) is applied to the control signal (Ũ_{I}) subsequent to the rectifier (RF).

12. The alternating current source of one of the claims 10 or 11 (FIG. 4a & 4b),
wherein the alternating current source encompasses a direct current (DC) voltage source, which provides a constant voltage (\overline{U}ₛ) to the first input of a differential amplifier (A₃),
and wherein the rectified control signal (\overline{U}_{I}) is connected to a second input of said differential amplifier (A₃),
and wherein the amplified differential signal at the output of said differential amplifier (A₃) is used to control the alternating signal generated by the generator.

13. The alternating current source of claim 12 (FIG. 4a & 4b),
wherein said amplified differential signal is connected to a controllable element (M)
and wherein said amplified differential signal controls the alternating voltage signal, which is generated by the generator.

14. The alternating current source of claim 13 (FIG. 4a & 4b),
wherein a multiplier (M) multiplies said amplified differential signal with the alternating voltage signal, which is generated by the generator, prior to serving as a control signal.

15. The alternating current source of claim 13 (FIG. 4a & 4b),
wherein the amplified differential signal is controlling a controllable amplifier,
and wherein said controllable amplifier amplifies the alternating voltage signal, which is generated by the generator.

16. The alternating current source of claim 13 (FIG. 4a & 4b),
wherein the controllable element is a linear optocoupler, incorporating a photo resistor or a photo transistor.

17. The alternating current source of one of the claims 13, 14, 15 or 16 (FIG. 4b),
wherein the output of the controllable element (M) is connected to a driving circuitry, in particular, via a resistor (R_{V}) to the inverting input of an amplifier (A₁),
wherein the non-inverting input of the amplifier (A₁) is connected to ground,
and wherein the output of the amplifier (A₁) is connected to the first terminal of the coil at the device side of the first transformer
and wherein the second terminal of the coil at the device side is connected to the inverting input of the amplifier (A₁).

18. An alternating current source for generating an alternating current (I) across the body of a patient (Z_{X}), comprising
A first transformer (T), which defines a patient side and a device side which is insulated from the patient side, and whose coil at the patient side provides a first and a second terminal, which can be connected to the patient, and
A generator on the device side, which generates an alternating voltage signal for controlling an alternating current source, wherein the alternating current source excites a coil at the device side upon connecting the first terminal and the second terminal via the patient as the load (Z_{X}),
wherein a voltage, which is measured across the coil at the device side of the transformer (T), is utilized for a positive feedback.

19. The alternating current source of claim 18 (FIG. 1),
wherein a terminal of the first coil of the transformer (T) at the device side is connected to the inverting input of a differential amplifier (A₀), and via a voltage divider (P1) to the non-inverting input and to the output of said differential amplifier (A₀).

20. The alternating current source of claim 19 (FIG. 2),
wherein a first terminal of the first coil of the transformer (T) at the device side is connected to the inverting input of a differential amplifier (A₀′), and a second terminal of the first coil of the transformer (T) at the device side is connected to the output of said differential amplifier (A₀′),
and wherein at least an additional, second coil at the device side is provided, which is not connected to the generator but to the non-inverting input of the differential amplifier (A₀′) in such a way that a voltage occurring across the second coil is fed to this input of the differential amplifier (A₀′).

21. The alternating current source of claim 20 (FIG. 2),
wherein the additional second coil is connected via a preferably variable resistor (P₂) to the non-inverting input of the differential amplifier (A₀′).

22. Method for generating an alternating current incorporating the following steps:
connection of a load (Z_{X}) to a coil at the patient side of a transformer (T¹),
Generating an alternating voltage,
Converting of the alternating voltage into an alternating current,
Excitement of a coil at the device side of the transformer (T₁) with the consequence of induction of an alternating current (I) in the coil at the patient side of the transformer (T₁),
Generating of a control signal, which is representative of the alternating current (I) at the patient side,
Providing said control signal to a control, which controls the alternating voltage or the alternating current.
